(19) United States
(12) Patent Application Publication
(10) Pub. No.: US 2019/0328912 A1
(43) Pub. Date: Oct. 31, 2019

(54) System and method for sterilizing and/or deimmunizing an object

(71) Applicant: ONEightyºC Technologies Corporation, Burlington, MA (US)

(72) Inventors: Kevin Staid, Lowell, MA (US); John Erickson, Middleton, MA (US); Theresa L. O'Keefe, Waltham, MA (US)

(21) Appl. No.: 15/330,469

(22) Filed: Sep. 23, 2016

Related U.S. Application Data
(60) Provisional application No. 62/232,055, filed on Sep. 24, 2015.

(51) Int. Cl. A61L 2/00; A61L 2/12; A61L 2/18; A61L 2/24; A61L 2/22

(52) U.S. Cl. CPC A61L 2/0064; A61L 2/0023; A61L 2/0088; A61L 2/22; A61L 2/18; A61L 2/24; A61L 2/12

(57) ABSTRACT

A system for sterilizing and/or deimmunizing an object includes a stationary chamber at ambient pressure configured to store an object to be sterilized and/or deimmunized therein. An electromagnetic device is coupled to the chamber and configured to direct microwaves at the object. A solvent spray subsystem is coupled to the chamber and configured to apply a solvent to the object such that the object is completely coated and/or saturated with the solvent. A controller subsystem coupled to the electromagnetic device and the solvent spray subsystem is configured to provide a cycle of activating the solvent spray subsystem for a predetermined amount of time, activating the electromagnetic device for a predetermined amount of time, and repeating the cycle a predetermined amount of times to irreversibly destroy proteins on the object to sterilize and/or deimmunize the object.

### RELATED APPLICATIONS

This application claims benefit of and priority to U.S. Provisional Application Ser. No. 62/232,055 filed Sep. 24, 2015, under 35 U.S.C. §§ 119, 120, 363, 365, and 37 C.F.R. § 1.55 and §1.78, which is incorporated herein by this reference.

### FIELD OF THE INVENTION

This invention relates to a system and method for sterilizing and/or deimmunizing an object.

COMPUTER PROGRAM LISTING APPENDIX

A computer program listing appendix is filed herewith on compact disk. The material on the compact disk is hereby incorporated by reference. Two identical compact disks have been submitted. Each compact disk contains files to be used with an appropriate integrated development environment (IDE), such as LabView®. The two disks were created on Sep. 15, 2016.

### BACKGROUND OF THE INVENTION

As medical treatments and diagnostics move away from traditional large incision processes, medical devices have become more flexible and complicated. To enable flexibility and/or smaller incisions, medical devices often use non-metal materials including a wide range of plastics, and the like. Additionally, the equipment frequently includes articulating joints and narrow lumens.

Medical devices composed of plastics with articulating joints and narrow lumens are frequently not robust enough to survive the rigors of conventional autoclave sterilization methods. As a result, they can only be prepared for reuse by vigorous cleaning and disinfection processes that frequently leave behind, including, inter alia, infectious and/or immunogenic agents defined herein as infectious proteins, spore forming bacteria, vegetative bacteria, funguses, infectious or immunogenic proteins, and toxic proteins that can infect and injure patients who are later treated using the insufficiently sterilized medical equipment.

Some of these infectious agents can be effectively eliminated through thorough cleaning and disinfection of medical equipment. Other infectious agents are extremely difficult to eliminate from medical equipment. The Center for Disease Control (CDC) lists examples of infectious agents and microorganisms by resistance to disinfection and sterilization processes. See Table 1 below:

TABLE 1
Decreasing order of resistance of infectious agents
and microorganisms to disinfection and sterilization

| Agent Category | Example Organisms or Diseases |
|---|---|
| Prions | Creutfeldt-Jakob Disease |
| Bacterial spores | Bacillius atrophaeus |
| Coccidica | Cryptosporidium |
| Mycobacteria | M. tuberculosis, M. terrae |
| Nonlipid or small viruses | polio, coxsackie |
| Fungi | Aspergillus, Candida |
| Vegetative bacteria | S. aureus, P. aeruginosa |
| Lipid of medium-sized viruses | HIV, herpes, hepatitis B |

### (CDC's Guideline for Disinfection and Sterilization in Healthcare Facilities, 2008)

Some infectious agents, such as the HIV virus, are easy to remove from medical equipment. Many, including vegetative bacteria are moderately difficult to eliminate. Other infectious agents, such as prions, can only be destroyed by extremely harsh conditions that damage and/or destroy modern medical equipment. Failure to eliminate infectious agents from medical equipment before use can put patients at extreme risk of injury and death.

Sterilization is a physical or chemical process that completely destroys or removes all forms of infectious agents from an object, including spore forming bacteria. Such spores allow the bacteria to resist high temperatures and other harsh conditions. Although measured in Sterility Assurance Levels (SAL), sterility is an absolute condition, i.e. either an item is sterile or it is not. Disinfection is a process that eliminates many or all infectious agents on instruments with the exception of bacterial spores. Disinfection is not absolute and is classified into three different levels: 1) High-Level Disinfection: kills all microorganisms with the exception of many bacterial spores through the use of chemical sterilants used for a shorter exposure period than would be required for sterilization, 2) Intermediate-Level Disinfection: may kill mycobacteria, vegetative bacteria, most viruses, and most fungi but do not necessarily kill bacterial spores, and 3) Low-level disinfection: may kill most vegetative bacteria, some fungi, and some viruses.

Although vegetative bacteria may be only moderately difficult to eliminate, many vegetative bacteria are still found to contaminate medical equipment after cleaning and disinfection. In addition to causing disease in patients, a number of species have been found to carry genes that allow the bacteria to grow and remain infectious even during the patients' treatment with antibiotics. Examples include, inter alia, Clostridium difficile (C. diff), a, CRE (Carbapenem-resistant Enterobacteriace) and MRSA (Methicillin-resistant Staphylococcus aureus) that are resistant to many antibiotics and in a medical setting can cause severe intestinal infection and life-threatening bloodstream infections, pneumonia and surgical site infections.

Prions (PrP—protease resistant proteins) are a unique category of transmissible infectious agent that causes a wide range of diseases including new variant Creutzfeldt-Jakob Disease. As Prions are only protein and do not include DNA or RNA, their destruction may be termed deactivation instead of sterilization. Prions are an abnormally folded protein (PrP_{sc}) that cause disease symptoms by promoting the unfolding of the normal protein (PrP_{c}) and refolding into the disease causing form (PrP_{sc}). With most infectious agents, conventional heat or steam systems and methods are sufficient to render the agents permanently non infections. However, such conventional heat and steam methods are unable to eliminate infectious prions from medical equipment.

When determining what level of sterilization or disinfection is appropriate for a particular reusable medical instrument or equipment, the Centers for Disease Control and Preventions (“CDCP”) uses a classification scheme which categorizes items, such as medical instruments and equipment, as either critical, semi-critical, or non-critical according to the degree of risk of infection being introduced by their use if not properly sterilized. Critical items represent the highest level of risk of infection if contaminated with any microorganism. Examples include medical instruments and equipment that enter tissue or the vascular system and include surgical instruments and equipment, cardiac and urinary catheters, implants and ultrasound probes used in body cavities. Medical instruments and equipment must by sterilized between uses. Semi-critical items, such as medical instruments and equipment, represent the next highest level of risk of infection are items that contact mucous membranes, such as the mucous membrane of the lungs or gastrointestinal tract. Semi-critical items are generally less likely to transfer common bacterial spores between patients but are highly susceptible to be able to transfer other organisms, such as bacteria, mycobacteria, and viruses. Semi-critical items require minimal high-level disinfection. While laparoscopes and arthroscopes should ideally be sterilized, they sometimes undergo a semi-critical level disinfection between patients.

Non-critical items, such as medical instruments and equipment that contact the skin but not mucous membranes, represent the least of risk for the transfer of infection between patients. Examples used in patient care include blood pressure cuffs, bedpans, crutches, and the like, and other related items.

Using a combination of one or more of heat, steam, water and microwaves to sterilize and/or disinfect medical equipment is known in the art, e.g., as disclosed in U.S. Pat. No. 6,900,421, incorporated herein by reference. The '412 Patent teaches a complicated and cumbersome system which must be pressurized by requiring a sealed first chamber capable of withstanding internal pressure and vacuum, generating steam greater than 1 atmosphere, introducing steam into the chamber, and removing the steam or by displacing it with nitrogen.

Another conventional apparatus for heating, disinfecting and sterilizing materials using microwave radiation, heat and water is disclosed in U.S. Pat. No. 5,879,643, incorporated by reference herein. As disclosed therein, a microwave device radiates microwave energy at refuse inside a container located in a chamber. The '643 Patent also teaches using a spray system with heated water which moistens the material being treated. The goal of the '643 Patent is to use water to eliminate the risk of fire which may result from using microwaves which may excessively heat the water.

U.S. Pat. Nos. 7,507,369, 7,687,045, and 7,939,016 now owned by the assignee hereof, teach another complicated and cumbersome system for disinfecting and/or sterilizing mail. As disclosed therein, mail to be disinfected is placed in a rotating drum and subjected to a source of radiation, microwaves, ultraviolet radiation, and a chemical decontamination unit.

All of the conventional systems discussed above which utilize one or more of microwaves, water, steam, and/or heat fail to teach or disclose irreversibly destroying proteins which are components of infectious and/or immunogenic agents, including, inter alia, bacterial spores, vegetative bacteria, viruses, funguses, infectious or immunogenic proteins, and toxic proteins to sterilize and/or deimmunize an object.

### SUMMARY OF THE INVENTION

In one aspect, a system for sterilizing and/or deimmunizing an object is featured. The system includes a stationary chamber at ambient pressure configured to store an object to be sterilized and/or deimmunized therein. An electromagnetic device coupled to the chamber is configured to direct microwaves at the object. A solvent spray subsystem coupled to the chamber is configured to apply a solvent to the object such that the object is completely coated and/or saturated with the solvent. A controller subsystem coupled to the electromagnetic device and the solvent spray subsystem is configured to provide a cycle of activating the solvent spray subsystem for a predetermined amount of time, activating the electromagnetic device for a predetermined amount of time, and repeating the cycle a predetermined amount of times to irreversibly destroy proteins on the object to sterilize and/or deimmunizing the object.

In one embodiment, the proteins are components of infectious and/or immunogenic agents which may include spore forming bacteria, vegetative bacteria, viruses, funguses, infectious or immunogenic proteins, and toxic proteins. The electromagnetic device and the chamber may be configured as a modified microwave oven. The electromagnetic device may be configured to generate the microwaves at a predetermined range of frequencies. The electromagnetic device may be configured to generate the microwaves at a desired frequency. The controller subsystem may be configured to control the amount of power provided by the electromagnetic device, a microwave output period, a duty cycle, and a mode for applying the microwaves. The mode may include pulse width modulation (PWM) and proportional integral derivative (PID). The controller subsystem may be configured to set the power of the electromagnetic device to about 1,000 watts to provide microwaves at a frequency of about 2.54 GHz. The system may include a mode stirrer coupled to the electromagnetic device. The solvent spray subsystem may include a reservoir for storing the solvent and a pump. The controller subsystem may be configured to control the pump such that the solvent spray subsystem applies the solvent to the object a predetermined amount of time. The solvent may include one or more of water, an ionic detergent and a non-ionic detergent that may assist in denaturing proteins such that they are most susceptible to destruction by the system. The controller may be configured to provide a cycle of activating the solvent spray subsystem for about 2 minutes, activating the electromagnetic device for about 4 minutes, and repeating the cycle 12 times to irreversibly destroy proteins on the object. The electromagnetic device may be activated for the predetermined amount of time to heat the chamber and the object to a predetermined range of temperatures including a desired temperature. The system may include one or more heating devices coupled to the chamber configured to heat an environment inside the chamber and the object to a predetermined range of temperatures including a desired temperature. The system may include a plurality of temperature sensors configured to measure temperature inside the chamber. The controller subsystem may be configured to provide a cycle of activating the solvent spray subsystem for a predetermined amount of time, activating the one or more heating devices for a predetermined amount of time to heat the chamber and the object to a predetermined range of temperatures including a desired temperature, activating the electromagnetic device for a predetermined amount of time, and repeating the cycle a predetermined number of times to irreversibly destroy proteins on the object to effectively sterilize and/or deimmunize the object.

In another aspect, a method for sterilizing and/or deimmunizing an object is featured. The method includes providing a stationary chamber at ambient pressure configured to store an object to be sterilized and/or deimmunized therein, directing microwaves at the object, and applying solvent to the object to completely coat and/or saturate the object with the solvent. The method also includes providing a cycle of applying the solvent for a predetermined amount of time, directing the microwaves at the object for a predetermined amount of time, and repeating the cycle a predetermined number of times to irreversibly destroy proteins on the object to sterilize and/or deimmunize the object.

In one embodiment, the proteins may be components of infectious and/or immunogenic agents including spore forming bacteria, vegetative bacteria, viruses, funguses, infectious or immunogenic proteins, and toxic proteins. The solvent may be applied to the object for about 2 minutes and the microwaves are applied to the object for about 4 minutes and the cycle may be repeated 12 times. The microwaves may be provided at a frequency of about 2.54 GHz. 22. The method may include heating an environment inside the chamber and the object to a predetermined range of temperatures including a desired temperature. The method may further include providing a cycle of applying solvent to the object for a predetermined amount of time, applying the heat for a predetermined amount of time, applying the microwaves for a predetermined amount of time, and repeating that cycle for a predetermined number of times to irreversibly destroy proteins on the object to sterilize and/or deimmunize the object.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a schematic block diagram showing the primary components of one embodiment of the system for sterilizing and/or deimmunizing an object;

FIG. 2 is a three-dimensional front view of the system shown in FIG. 1 configured as a modified microwave oven;

FIG. 3 is a three-dimensional view showing the inside of the modified microwave oven shown in FIG. 2;

FIG. 4 is a schematic circuit diagram showing in further detail the primary components of the controller subsystem shown in FIGS. 1 and 2;

FIG. 5 is a screen shot showing one example of the various parameters controlled by the controller subsystem shown in FIGS. 1 and 2;

FIG. 6 is a flow chart showing the primary steps associated with one embodiment of the method for sterilizing and/or deimmunizing an object;

FIGS. 7A and 7B show an example of a filter strip having an infectious and/or immunogenic agent thereon to be sterilized and/or deimmunized using the system and method shown in FIGS. 1-6;

FIG. 8 shows an example of a Western Analysis for sample shown in FIG. 7B sterilized and/or deimmunized using the system and method shown in FIGS. 1-6:

FIG. 9 shows an example of another filter strip having a different infectious agent thereon to be sterilized and/or deimmunized using the system and method shown in FIG. 1-6; and

FIG. 10 is a three-dimensional front view showing examples of containment chambers which may he placed inside the chamber shown in FIGS. 1-3 to store objects to be sterilized and/or deimmunized therein.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

As discussed in the Background section above, conventional systems and methods which utilize one or more of microwaves, water, steam, and heat to sterilize and/or disinfect objects or medical equipment are complicated cumbersome systems which are pressurized, heat the water before it is applied, require a container inside a chamber to place the material to be sterilized or disinfected therein, use water to extinguish any fires that may result from using microwaves for decontamination and/or sterilization, or rely on rely on a tumbling drum. Such systems fail to teach irreversibly destroying proteins to sterilize and/or deimmunize an object.

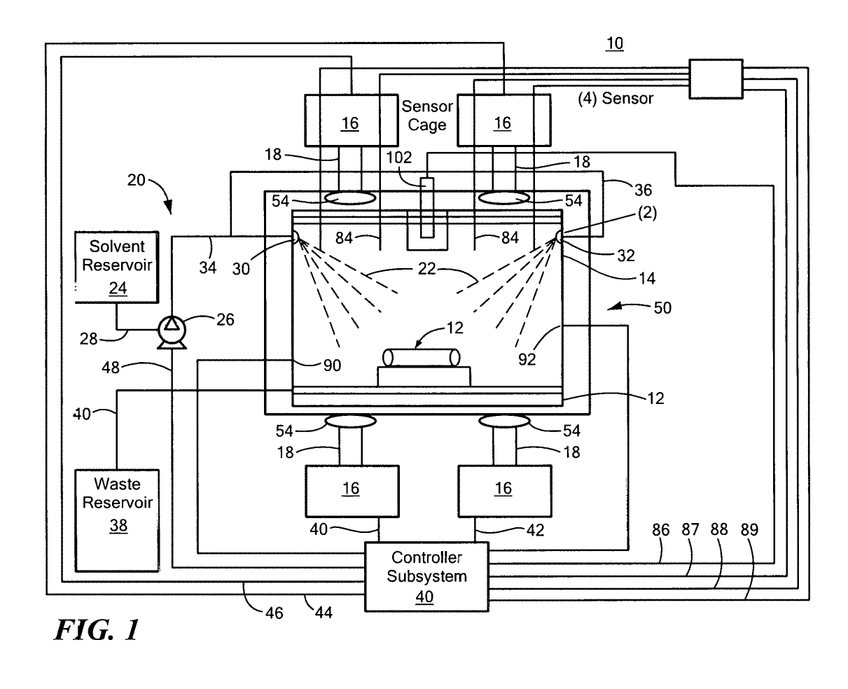

There is shown in FIG. 1, one embodiment of system 10 for sterilizing and/or deimmunizing object 12 that has been exposed to and has infectious and/or immunogenic agents thereon including, inter alia, infectious proteins, including priors and similar type infectious proteins, viruses, spore forming bacteria, vegetative bacteria, funguses, infectious or immunogenic proteins and, toxic proteins. As defined herein, object 12 to be sterilized and/or deimmunized may include medical equipment, and surgical equipment, medical devices, surgical instruments, dental equipment, devices, and instruments, veterinary equipment, devices, and instruments, or any object or thing that needs to be sterilized and/or deimmunized. System includes stationary chamber 14 at ambient pressure and configured to store object 12 to be sterilized and/or deimmunized therein. System 10 also includes an electromagnetic device coupled to the chamber to direct microwaves at the medical equipment. In one example, the electromagnetic device may include four magnetrons 16 each with waveguide 18 coupled to chamber 14 as shown. In other examples, there may be more or less than four magnetrons 16 each with an associated waveguide 18. Preferably, the length of the microwaves provided by magnetron 16 with waveguide 18 is centered about a predetermined range of microwaves frequencies, e.g., between about 900 MHz and about 30 GHz or at a centered at a desired frequency. In one example, the power of magnetron 16 can be controlled, e.g., set at a desired power level, such that each magnetron 16 with waveguide 18 generates microwaves a frequency centered at about 2.45 GHz. In other examples, the power of magnetrons 16 can be set such that the frequency of the microwaves may be centered higher or lower than 2.45 GHz.

System 10 also includes solvent spray subsystem 20 coupled to chamber 14 configured to apply solvent 22 to object 12 to be sterilized and/or deimmunized such that object 12 is completely coated and/or saturated with solvent 22. In one example, solvent 22 may be at ambient temperature. In other designs, solvent 22 may be heated or cooled to improve sterilization and/or deimmunization as needed. In one example, solvent spray subsystem 20 includes solvent reservoir 24 which stores solvent 22 and pump 26 coupled to solvent reservoir 22 by line 28. Pump 26 delivers solvent 22 by line 34 to solvent atomizer 30 and/or by line 36 to solvent atomizer 32. Solvent spray subsystem 22 may also include waste reservoir 38 coupled to chamber 14 by line 40 which recovers solvent 22 directed at object 12 to be sterilized and/or deimmunized. Solvent 22 may be water, an ionic detergent and/or a non-ionic detergent or a combination thereof, e.g., Sodium dodecyl sulfate (SDS, also called sodium lauryl sulfate), Tween (Polysorbate), Triton X-100 (a nonionic surfactant that has a hydrophilic polyethylene oxide chain and an aromatic hydrocarbon lipophilic or hydrophobic group), NP-40 (nonyl phenoxypolyethoxylethanol), octyl glucoside, non-detergent sulfobetaines, mild acids and bases, hydrogen peroxide, biostatic, antimicrobial, and fungicide elements including copper, nickel, iodine, zinc, silver, gold, tin and lead. When solvent 22 is an ionic detergent or non-ionic detergent, it preferably supports denaturation of the contaminating agents or proteins such that they are more susceptible to sterilization and/or deimmunization by system 10.

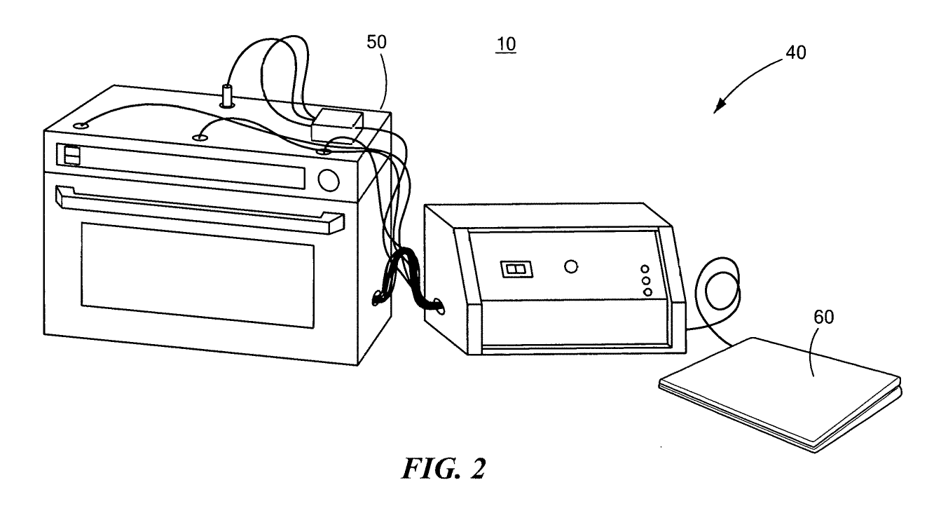

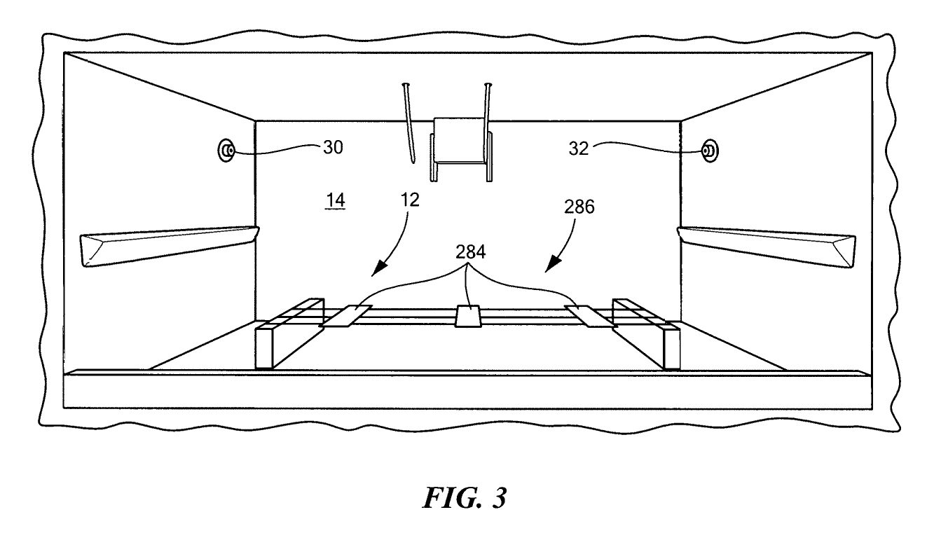

System 10 also includes controller subsystem 40 coupled to the electromagnetic device comprised of one or more magnetrons 16 with waveguide 18 by lines 40, 42, 44 and 46 and solvent spray subsystem 20 by line 48. Controller subsystem 40 is configured to provide a cycle of activating solvent spray subsystem 20 for a predetermined amount of time, activating the electromagnetic device for a predetermined amount of time, and repeating the cycle a predetermined number of times to irreversibly destroy proteins on object 12 to effectively sterilize and/or deimmunize object 12. As used herein, the proteins irreversibly destroyed may be isolated proteins and/or proteins within tissue, a biomass, or an organism. The proteins on object 12 are components of infectious and/or immunogenic agents including, inter alia, spore forming bacteria, vegetative bacteria, viruses, funguses, infectious or immunogenic proteins, and toxic proteins. In one example, solvent spray subsystem 20 is activated for 2 minutes to completely saturate or coat object 12 with solvent 22. In this example, solvent 22 is water. The electromagnetic device is then activated for 4 minutes at 1,000 watts to provide microwaves at a frequency of about 2.450 GHz. The cycle is then repeated 12 times to completely and irreversibly destroy proteins on object 12 to sterilize and/or deimmunize object 12. In one example, the microwaves generated by the electromagnetic device 16 increased the temperature inside chamber 14 to a predetermined range of temperatures, e.g. from about 20° C. to about 140° C., and to a desired temperature, e.g., about 100° C. In other examples, the amount of time the solvent is applied may be more or less than 2 minutes and the amount of time the electromagnetic device is activated may be more or less than 4 minutes to efficiently sterilize and/or deimmunize object 12. The number of cycles of activating solvent spray subsystem 20 and the electromagnetic device may be more or less than 12 cycles, e.g., 1 cycle, 4 cycles, 8 cycles, 12 cycles, 16 cycles, or any number of desired cycles to effectively sterilize and/or deimmunize object 12. In one example, system 10 may be configured as modified microwave oven 50 as shown. In one example, modified microwave oven 50 may be a microwave oven available from Microwave Research & Applications, Inc, Carol Stream, Ill. 60188, which has been modified as shown in FIG. 1. FIG. 2, where like parts have been given like numbers, shows three-dimensional view of system 10 configured as modified microwave oven 50 with controller subsystem 40 which preferably includes computer subsystem 60, e.g., a general purpose computer, a laptop, personal computer, or similar type computing device. FIG. 3 shows a view of the inside of modified microwave oven 50 and shows in further detail examples of atomizers 30 and 32 of solvent spray subsystem 20, FIG. 1.

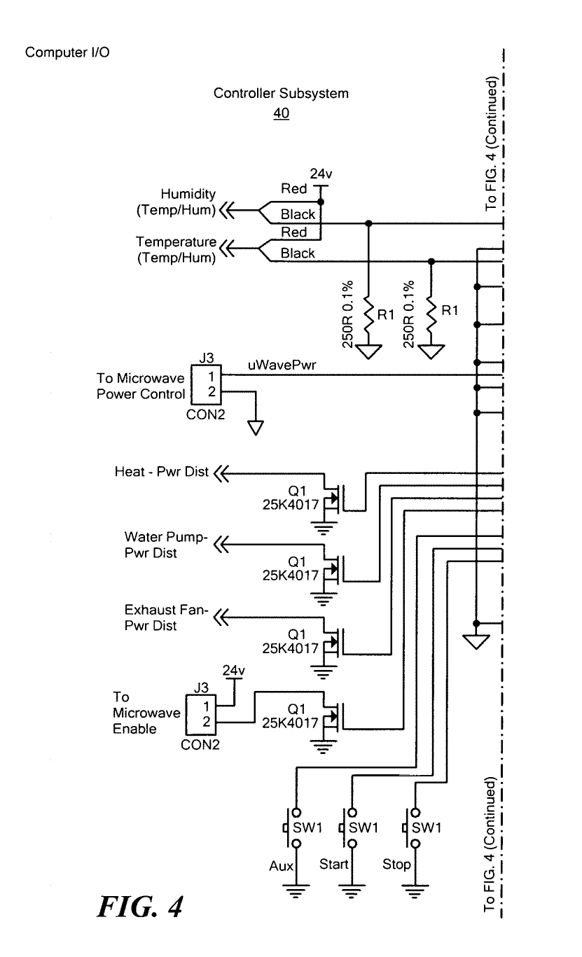

Controller subsystem 40, FIGS. 1 and 2, may include one or more processors, ASIC, firmware, hardware, and/or software (including firmware, resident software, micro code, and the like) or a combination of both hardware and software which may be part of controller subsystem 40. FIG. 4 shows a schematic circuit diagram showing in further detail the primary components of controller subsystem 40 which, in this example, includes microprocessor 100, laptop computer 60, and the associated connections to the temperature sensors 84, FIG. 1 (discussed below), microwave control, humidity sensor 102, and the like as shown.

Any combination of computer-readable media or memory may be utilized for controller subsystem 40. The computer-readable media or memory may be a computer-readable signal medium or a computer-readable storage medium. A computer-readable storage medium or memory may be electronic, magnetic, optical, electromagnetic, infrared, or semiconductor system, apparatus, or device, or any suitable combination thereof. Other examples may include an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing.

Computer program code for the one or more programs for carrying out the instructions or operation of one or more embodiments of controller subsystem 40 may be performed in an appropriate IDE, such as LabView® or similar IDE or may be written in any combination of one or more programming languages, including an object oriented programming language, e.g., C++, Smalltalk, Java, and the like, and conventional procedural programming languages, such as the “C” programming language or similar programming languages.

These computer program instructions may be provided to a processor of a general purpose computer, a controller, processor, or similar device included as part of controller subsystem 40, or separate from controller subsystem 40, or other programmable data processing apparatus to produce a machine, such that the instructions, which execute via the processor of the computer or other programmable data processing apparatus, create means for implementing the functions/acts specified in the flowchart and/or block diagram block or blocks.

The computer program instructions may also be stored in a computer-readable medium that can direct a computer, other programmable data processing apparatus, or other devices to function in a particular manner, such that the instructions stored in the computer-readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer-implemented process such that the instructions which execute on the computer or other programmable apparatus provide processes for implementing the functions/acts specified in the flowchart and/or block diagram block or blocks.

For enablement purposes only, the Computer Program Listing Appendix provided herewith can be executed on one or more processors ASIC, firmware, hardware, or general purpose computer in an appropriate IDE to carry out the primary steps and/or functions of controller subsystem 40 as discussed above and recited in the Claims hereof. Other equivalent algorithms and code can be designed by a software engineer or programmer skilled in the art using the information provided herein.

Preferably, controller subsystem 40 controls the amount of power provided by the one or more magnetrons 16 of the electromagnetic device, a microwave output period, a duty cycle, and a mode for applying the microwaves. The microwave output period and the duty time determines the amount of time the electromagnetic device is ON and OFF. The duty cycle works in conjunction with the period. During a period, magnetrons 16, FIG. 1, are is ON for the On time, then OFF for the Off time. This repeats for the duration of the sterilization and/or deimmunization process of system 10 and the method thereof. The duty cycle identities the percentage of time the microwave is ON.

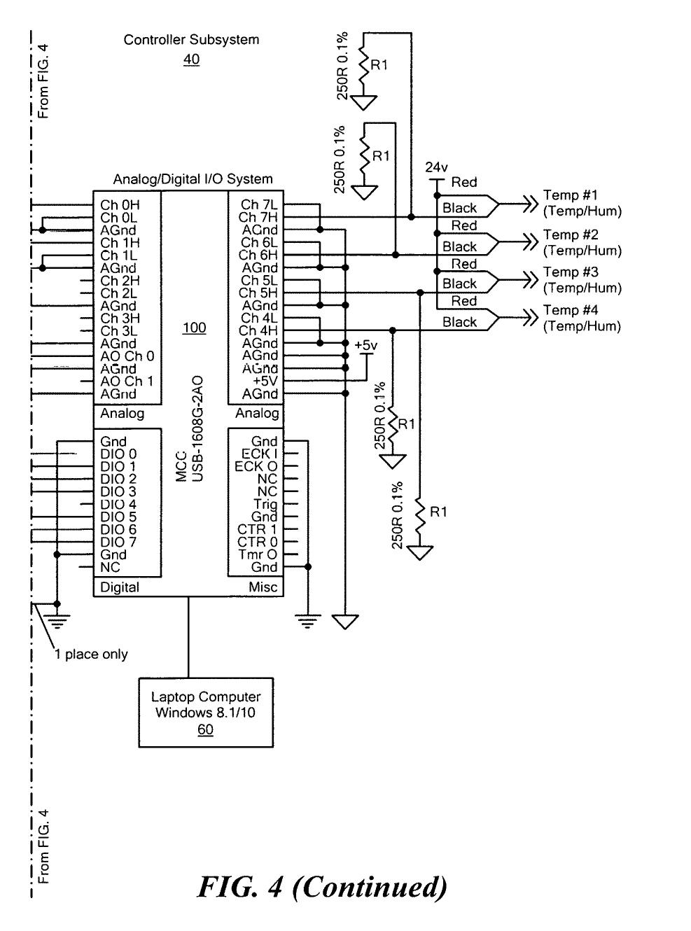

For example, FIG. 5 shows one embodiment of control interface screen 58 generated by computer subsystem 60, FIG. 2, of controller subsystem 40, FIGS. 1 and 2. In this example, control interface screen 58, FIG. 5, allows a user to set the power provided to one or more magnetrons 16, FIG. 1, and the resulting frequency of the microwaves using control buttons 62, the microwave output period using control button 64, the duty cycle using control buttons 66, and the mode using control buttons 70. In one example, the mode may include pulse width modulation (PWM) or proportional integral derivative (PID).

In one example, to provide the cycle of activating the activating solvent spray subsystem 20, FIG. 1, for a predetermined amount of time, the duty cycle is programmed (0% to 100%) using duty cycle control buttons 164 and the output period is set, e.g., to 60 seconds, using output period controls 162. The mode is set to PWM using mode control 166.

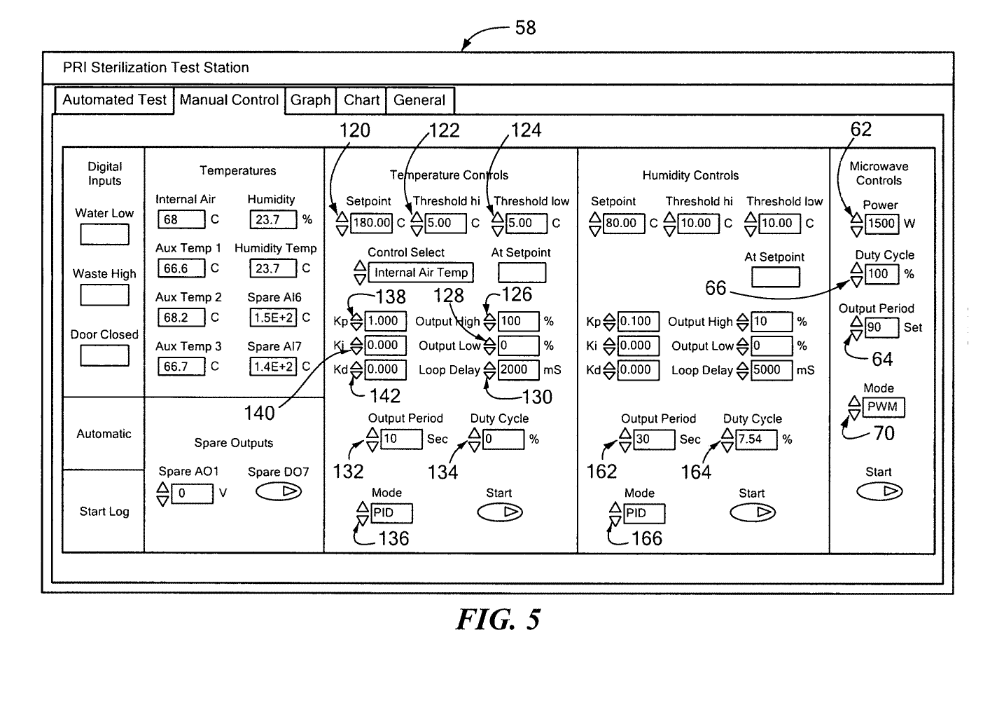

The method for sterilizing and/or deimmunizing an object of one embodiment of this invention includes providing a stationary chamber at ambient pressure configured to store an object to be sterilized and/or deimmunized therein, step 200, FIG. 6, directing the microwaves at the object inside the chamber, step 202, and applying solvent to the object to be sterilized and/or deimmunized to completely saturate and/or coat the object with the solvent, step 204. The method also includes providing a cycle of applying the solvent for a predetermined amount of time, directing the microwaves at the object for a predetermined amount of time, and repeating the cycle a predetermined number of times to irreversibly destroy proteins on the object to sterilize and/or deimmunize the object, step 206.

The following example is meant to illustrate and not limit the present invention.

### EXAMPLE

### Destruction of Proteins

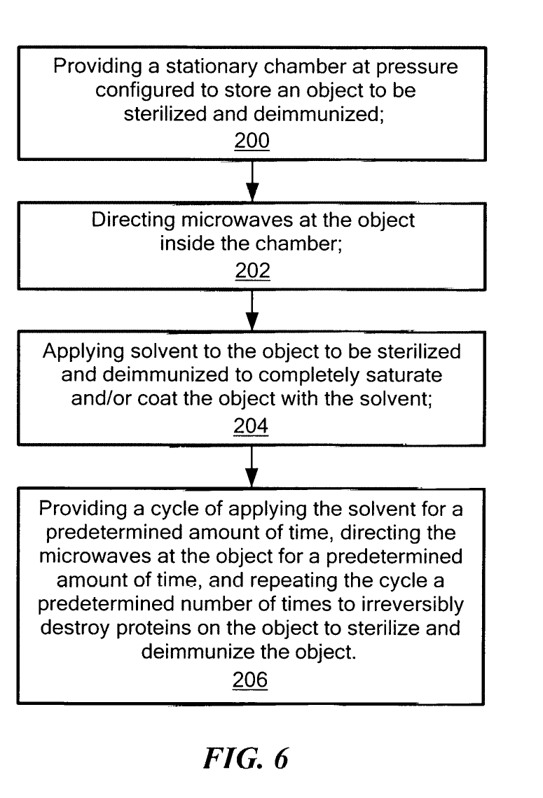

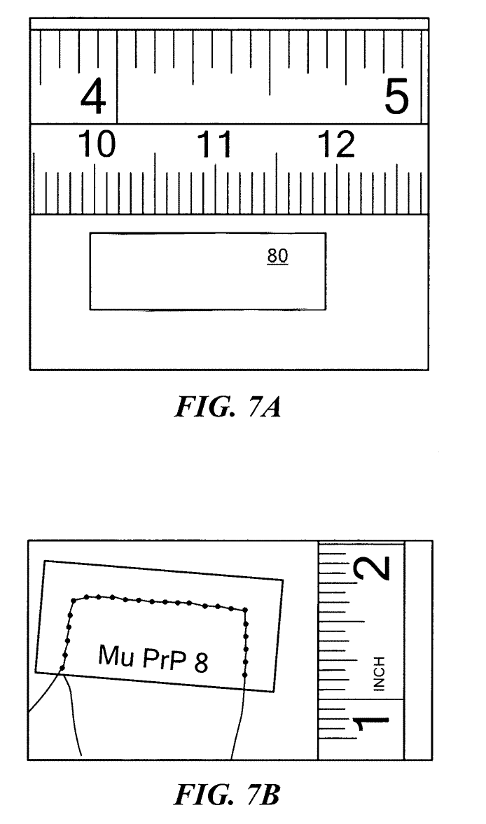

Experiments were conducted to demonstrate that a combination of vaporizing solvent, electromagnetic radiation, e.g., microwaves, and the heat generated by the microwaves which heats the environment inside the chamber and the object to be sterilized and/or deimmunized to a predetermined range of temperature including a desired temperature, irreversibly destroyed proteins to effectively sterilize and/or deimmunize an object having an infectious and/or immunogenic agent thereon. In this example, a stable PrP protein was selected for the experiments as it cannot be irreversibly destroyed using a standard autoclave. For the experiments, filter paper was cut into a strip, e.g., strip 80, FIG. 7A, and samples were created that each contained about 1 ug of a structurally robust mouse PrP protein and wrapped in 100% cotton paper and sewed in place as shown in FIG. 7B to avoid extraneous contamination. This containment was placed in a second layer of 100% cotton paper to increase stability during treatment. The samples were treated with system 10 and the method thereof as discussed above for differing number cycles of applying moisture saturation and microwaves. After treatment, the samples were subjected to standard Western blot analysis. For this, the samples were suspended in loading buffer, boiled to denature the proteins and run on a denaturing protein gel to separate intact protein and substantially intact proteins from small polypeptides and amino acids. The samples were then transferred to a nylon membrane. The membranes were then incubated with a primary antibody that specifically binds to a region near the C-terminus of the protein and visualizes with a secondary HRP-labelled antibody. The process enables the detection of any intact or partially intact protein sample. Proteins irreversibly broken into small polypeptides and amino acids and destroyed will not be visualized. With the Western Blot analysis, it was possible to see that certain combinations of the treatment cycles of system 10 and the method thereof as discussed above with reference to one or more of FIGS. 1-6 irreversibly and completely destroyed the protein samples, shown by the absence of protein bands of gel as shown by lane 5, indicated at 82, FIG. 8. Other treatment conditions did not destroy the highly robust proteins.

### Example 2

### Additional Parameter Testing

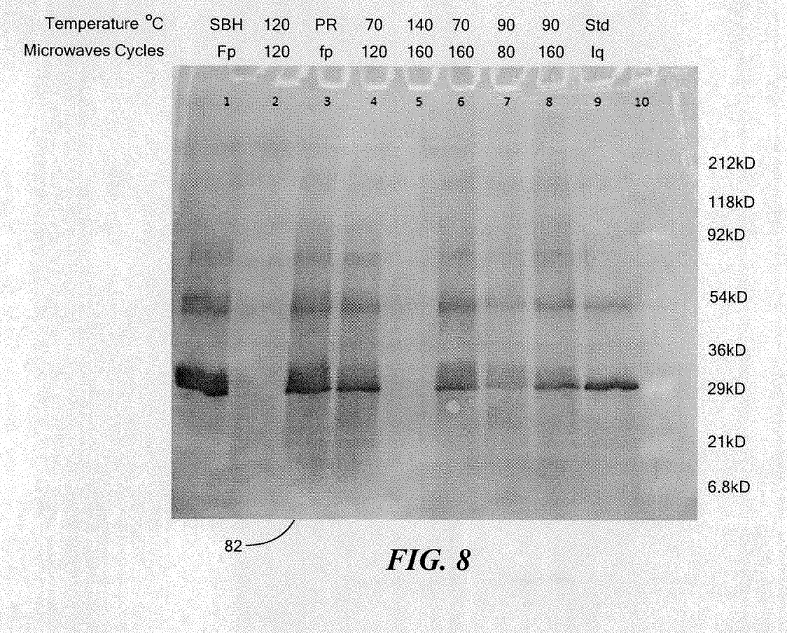

Testing was conducted to determine the ability of one or more embodiments of system 10 and the method thereof to sterilize and/or deimmunize object 12 material using biologic indicator strips impregnated with a set number of biologic spores. For the procedure, biologic indicator strips 284, having infectious agent thereon, FIG. 9, were placed in chamber 14, FIGS. 1 and 3, e.g., indicated at 286, FIG. 3, and subjected to a predetermined number of cycles of applying solvent and microwaves using system 10 and the method thereof as discussed above. After the sterilization cycles, biologic indicator strip 286 were placed in culture media and incubated for 10 days. If bacteria grow during that period, sterilization failed. Only if no bacteria grew during the 10 day culture system 10 and the method thereof be qualified for sterilization.

Depending on the sterilization technology being used, one of three spore forming bacteria is used to determine successful sterilization. To qualify steam sterilization (autoclave), the bacterial species G. stearothermophilus (10⁴-10⁶ spores) was used. For Gamma radiation sterilization, the bacterial species B. pumilus (10⁴-10⁶ spores) was used. For Ethylene Oxide (ETO) sterilization, the bacterial species B. atrophaeus (10⁴-10⁶ spores) was used. B. atrophaeus is the standard surrogate species for anthrax (pathogenic B. anthracis).

For the experiments using standard biologic indicator strips 284, FIG. 9, the temperature of chamber 14 and object 12 was varied, e.g., about 60° C. to about 160° C. The saturation of object 12 and the power and cycle of the electromagnetic device were applied as discussed above with reference to one or more of FIGS. 1-6. System 10 and the method thereof was able to sterilize G. stearothermophilus and B. pumilus spores at between about 100° C. and about 120° C. System 10 and the method thereof was able to sterilize B. atrophaeus at between 120° C. and 140° C.

Additional testing was conducted using the bacterial species B. atrophaeus (anthrax surrogate). For this experiment, the number of spores inserted was logarithmically increased from 10⁴-10⁶ spores to the extremely high number of 10⁻¹⁰ spores. The test was run at 150° C. and 80 cycles. System 10 and the method thereof was able to successfully kill all the spores.

The result is system 10 and the method thereof completely and irreversibly destroys proteins on an object to efficiently and effectively sterilize and/or deimmunize any object that needs to be sterilized and/or deimmunized. System 10 effectively irreversibly destroys proteins that are components of infectious and/or immunogenic agents including spore forming bacteria, vegetative bacteria, viruses, funguses, infectious or immunogenic proteins, and toxic proteins that may be found on an object to be sterilized and/or deimmunized. System 10 is easy to use, does not need to be pressurized, and does not require using a container inside the chamber. System 10 and the method thereof is also much less complex than the conventional systems discussed in the Background section above. The proteins irreversibly destroyed by system 10 includes prions which are a unique category of transmissible infectious agents that cause a wide range of diseases.

In one embodiment, system 10, FIG. 1, may include mode stirrer 54 coupled between waveguide 18 and chamber 14 as shown to provide a more uniform distribution of the microwaves generated by magnetron 16 and waveguide 18.

System 10, FIG. 1, may also include a plurality of temperature sensors 84 coupled to controller subsystem 40 by lines 86, 87, 88 and 89 as shown configured to measure the temperature inside chamber 14.

In one design, instead of utilizing the electromagnetic device discussed above with reference to FIG. 1 to heat inside the environment inside chamber 14 and object 12 to be sterilized and/or deimmunized to the predetermined range of temperatures, e.g., 20° C. to 140° C., e.g., about 100° C., system 10 may also include one or more heaters, e.g., heaters 90, 92 coupled to the walls of chamber 14 as shown to heat the environment inside chamber 14 and object 12 to be sterilized and/or deimmunized to the predetermined range of temperatures including a desired temperature.

In one design, controller subsystem 40 may include temperature set point controls 120, FIG. 5, threshold high controls 122, threshold low controls 124, output high controls 126, output low controls 128, loop delay controls 130, output period controls 132, duty cycle controls 134, and mode controls 136, Kp (proportional coefficient) controls 138, Ki (integral coefficient) controls 140, and Kd (derivative coefficient) controls 142, of which one or more may be utilized to act the temperature parameters provided by heaters 90, 92, FIG. 1 to heat the environment inside chamber 14 and object 12 to the predetermined range of temperatures or a. desired temperature

In one example, controller subsystem 40 may be configured to provide a cycle of activating solvent spray subsystem 20 for a predetermined amount of time, activating heating devices 90, 92 for a predetermined amount of time, and activating the electromagnetic device a predetermined amount of time, and repeating the cycle a predetermined amount of times to irreversibly destroy proteins on object 12 to sterilize and/or deimmunize object 12.

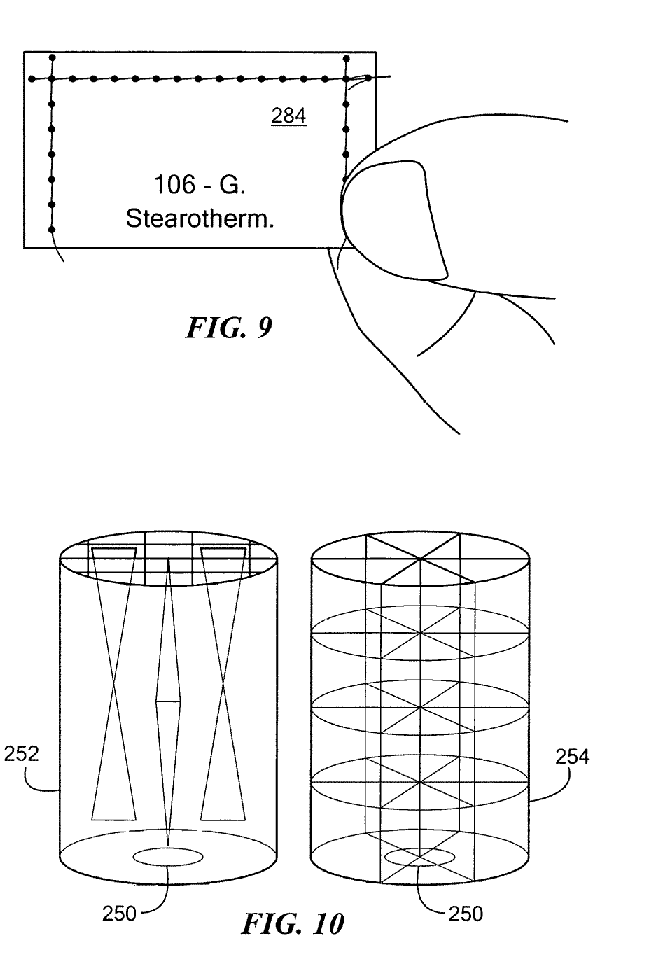

In one design, system 10 may include rotating cog 250, FIG. 10, which is preferably coupled to the floor of chamber 14 shown in FIGS. 1 and 3. In this design, system 10 also includes a containment chamber 252 or (containment chamber) 254 coupled to cog 250. Containment chamber 252 is designed to store larger medical equipment to be sterilized and or deimmunized and containment chamber 254 is designed to store smaller medical equipment to be sterilized and/or deimmunized as shown.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant can not be expected to describe certain insubstantial substitutes for any claim element amended.

What is claimed is:

1. A system for sterilizing and/or deimmunizing an object, the system comprising:
a stationary chamber at ambient pressure configured to store an object to be sterilized and/or deimmunized therein;
an electromagnetic device coupled to the chamber configured to direct microwaves at the object;
a solvent spray subsystem coupled to the chamber configured to apply a solvent to the object such that the object is completely coated and/or saturated with the solvent; and
a controller subsystem coupled to the electromagnetic device and the solvent spray subsystem configured to provide a cycle of activating the solvent spray subsystem for a predetermined amount of time, activating the electromagnetic device for a predetermined amount of time, and repeating the cycle a predetermined amount of times to irreversibly destroy proteins on the object to sterilize and/or deimmunizing the object.

2. The system of claim 1 in which the proteins are components of infectious and/or immunogenic agents including spore forming bacteria, vegetative bacteria, viruses, funguses, infectious or immunogenic proteins and toxic proteins.

3. The system of claim 1 in which the electromagnetic device and the chamber are configured as a modified microwave oven.

4. The system of claim 1 in which the electromagnetic device is configured to generate the microwaves at a predetermined range of frequencies.

5. The system of claim 4 in which the electromagnetic device is configured to generate the microwaves at a desired frequency.

6. The system of claim 1 in which the controller subsystem is configured to control the amount of power provided by the electromagnetic device, a microwave output period, a duty cycle, and a mode for applying the microwaves.

7. The system of claim 6 in which the mode includes pulse width modulation (PWM) and proportional integral derivative (PID).

8. The system of claim 6 in which the controller subsystem is configured to set the power of the electromagnetic device to about 1,000 watts to provide microwaves at a frequency of about 2.54 GHz.

9. The system of claim 1 further including a mode stirrer coupled to the electromagnetic device.

10. The system of claim 1 in which the solvent spray subsystem includes a reservoir for storing the solvent and a pump.

11. The system of claim 10 in which the controller subsystem is configured to control the pump such that the solvent spray subsystem applies the solvent to the object a predetermined amount of time.

12. The system of claim 1 in which the solvent includes one or more of water, an ionic detergent and a non-ionic detergent.

13. The system of claim 1 in which the controller is configured to provide a cycle of activating the solvent spray subsystem for about 2 minutes, activating the electromagnetic device for about 4 minutes, and repeating the cycle 12 times to irreversibly destroy proteins on the object.

14. The system of claim 1 in which activating the electromagnetic device for the predetermined amount of time heats the chamber and the object to a predetermined range of temperatures including a desired temperature.

15. The system of claim 1 further including one or more heating devices coupled to the chamber configured to heat an environment inside the chamber and the object to a predetermined range of temperatures including a desired temperature.

16. The system of claim 15 further including a plurality of temperature sensors configured to measure temperature inside the chamber.

17. The system of claim 15 in which the controller subsystem is configured to provide a cycle of activating the solvent spray subsystem for a predetermined amount of time, activating the one or more heating devices for a predetermined amount of time to heat the chamber and the object to a predetermined range of temperatures including a desired temperature, activating the electromagnetic device for a predetermined amount of time, and repeating the cycle a predetermined number of times to irreversibly destroy proteins on the object to effectively sterilize and/or deimmunize the object.

18. A method for sterilizing and/or deimmunizing an object, the method comprising:
providing a stationary chamber at ambient pressure configured to store an object to be sterilized and/or deimmunized therein;
directing microwaves at the object;
applying solvent to the object to completely coat and/or saturate the object with the solvent; and
providing a cycle of applying the solvent for a predetermined amount of time, directing the microwaves at the object for a predetermined amount of time, and repeating the cycle a predetermined number of times to irreversibly destroy proteins on the object to sterilize and/or deimmunize the object.

19. The method of claim 18 in which the proteins are components of infectious and/or immunogenic agents including spore forming bacteria, vegetative bacteria, viruses, funguses, infectious or immunogenic proteins, and toxic proteins.

20. The method of claim 18 in which the solvent is applied to the object for about 2 minutes and the microwaves are applied to the object for about 4 minutes and the cycle is repeated 12 times.

21. The method of claim 18 in which the microwaves are provided at a frequency of about 2.54 GHz.

22. The method of claim 18 further including heating an environment inside the chamber and the object to a predetermined range of temperatures including a desired temperature.

23. The method of claim 22 further including providing a cycle of applying the solvent to the object for a predetermined amount of time, applying heat for a predetermined amount of time, applying the microwaves for a predetermined amount of time, and repeating that cycle for a predetermined number of times to irreversibly destroy proteins on the object to sterilize and/or deimmunize the object.
